(19) United States
(12) Patent Application Publication
(10) Pub. No.: US 2010/0297647 A1
(43) Pub. Date: Nov. 25, 2010

(54) POLYMERASE-BASED SINGLE-MOLECULE SEQUENCING

(75) Inventor: Achillefs Kapanidis, Oxford (GB)

Correspondence Address: SPECKMAN LAW GROUP PLLC, 1201 THIRD AVENUE, SUITE 330, SEATTLE, WA, 98101, US

(73) Assignee: ISIS INNOVATION LIMITED, Oxford (GB)

(21) Appl. No.: 12/740,525

(22) PCT Filed: Oct. 30, 2008
(86) PCT No.: PCT/GB2008/003669

§ 371 Date: May 13, 2010

(30) Foreign Application Priority Data
Oct. 30, 2007 (GB) 0721340.8

(51) Int. Cl. C12Q 1/68; C12N 9/12

(52) U.S. Cl. 435/6; 435/194

(57) ABSTRACT

The invention relates to an assay method for determining the base sequence in a nucleic acid sample which comprises detection of a single base in said sequence using a polymerase modified to include at least one fluorophore and a dark quencher group; detecting and deducing the amount of energy transfer.

### FIELD OF THE INVENTION

This invention relates to a novel fluorescence spectroscopy method and assay platform for performing nucleic acid sequencing on individual nucleic acid molecules by using the activity of nucleic acid polymerases (polymerase-based single-molecule sequencing).

### BACKGROUND TO THE INVENTION

Sequencing the first human genome opened a new era in biology, biomedicine, and health. This feat required an intense worldwide effort that lasted over ten years and cost over 2 billion US dollars. Recently, new technologies such as the ones offered by Solexa/Illumina, Roche/454 Life Sciences, and ABI (known as the “second generation” of sequencing technology) have increased the speed and reduced the cost of human genome sequencing by orders of magnitude; the cost has reached approximately 100,000 US dollars in 2008. These figures clearly indicate that sequencing is still a very expensive approach for studying large populations of individuals, for using sequencing for pharmacogenomics and personalised medicine, for monitoring changes in the genome of an individual during the course of his lifetime (genome re-sequencing); for identifying binding sites for proteins (such as with the ChipSeq method); for discovering sequences coding for new genes and small RNAs; and for comparing genomes of healthy cells with those of tumour cells. To make such applications more affordable, new sequencing technologies are necessary; most of these technologies involve sequencing of individual DNA molecules and are often referred to as the “third generation” of sequencing technology. Development of such technologies has been encouraged by many national agencies, including a US National Institutes of Health initiative which aims to develop by 2015 methods that can sequence the human genome in a single day with 99.9% accuracy and at a cost of only 1000 US dollars.

Several of the existing DNA sequencing methods are based on the activity of nucleic acid polymerases, proteins that read information from a nucleic acid template (DNA or RNA) and copy it to a new nucleic acid strand. For example, DNA polymerases (DNAPs) read the information in a DNA template and copy it to a new DNA strand; RNA polymerases (RNAPs) read the information in a DNA template and copy it to a new RNA strand. DNAPs are more commonly used for sequencing; e.g., the Sanger DNA sequencing method (the main method used for completing the first human genome sequencing) was based on the activity of DNAP to incorporate di-deoxy-nucleotides (that act as chain terminators) on a new DNA strand. DNAP-based sequencing requires a DNA template and a DNA primer for the initiation of the copying process.

RNAPs have been used less extensively for sequencing; the RNAP-based mode of sequencing is known as transcriptional sequencing. Transcriptional sequencing has been used in the past at the bulk level (i.e., where billions of molecules are probed simultaneously); it relies on well characterised single-subunit phage RNAPs (e.g., bacteriophage T7 or SP6 RNAP) commonly used for in vitro synthesis of RNA. Transcription requires promoter DNA (which can be introduced using PCR or ligation reactions); however, in contrast to sequencing based on DNA polymerases, it does not require an initiation primer. Moreover, transcriptional sequencing is compatible with several sequencing reads per single DNA molecule since several RNAPs can operate on the same DNA simultaneously, thus increasing the number of potential reads per molecule and the accuracy of the recovered sequence. Recently, proof-of-principle single-molecule experiments using Escherichia coli RNAP using high-resolution optical tweezers instrumentation were reported, showing the feasibility of single-molecule transcriptional DNA sequencing.

Here we describe a DNA-sequencing strategy that uses real-time template-directed nucleic acid synthesis by nucleic-acid polymerases (a “sequencing-by-synthesis” approach). Specifically, we describe an implementation using single-RNA synthesis by an RNA polymerase. We note that the main concept of the strategy is fully compatible with sequencing based on other nucleic-acid polymerases, such as DNAP and reverse transcriptase. We describe both a single-colour and a multi-colour scheme for assigning each incorporated base. Both approaches require surface-immobilisation and total-internal-reflection fluorescence (TIRF) microscopy, technologies available in several laboratories, including the Kapanidis' group. Moreover, both approaches are based on efficient incorporation of 5′-modified nucleoside triphosphates (NTPs) carrying quenchers at the gamma- (or beta-) phosphate of each NTP; the quenchers permit fluorescence-aided base assignment and are released at the end of each incorporation cycle.

U.S. Pat. No. 7,052,847 describes a method of sequencing a target nucleic acid molecule comprising providing a mixture of the target nucleic acid molecule, a complementary primer, a nucleic acid polymerizing enzyme, and a plurality of types of nucleotides to be incorporated into a growing nucleic acid strand at an active site; and subjecting the mixture to a polymerization reaction under conditions suitable for formation of the growing nucleic acid strand by template-directed extension of primer; and optically identifying a time sequence of incorporation of nucleotides into the growing nucleic acid strand.

US Patent Application No. 2005/2148849 describes a method for determining the sequence of a polynucleotide, comprising the steps of reacting a target polynucleotide with an enzyme that is capable of interacting with and processing along the polynucleotide, under conditions sufficient to induce enzyme activity and detecting conformational changes in the enzyme as the enzyme processes along the polynucleotide. The detection of a conformational change is carried out by measuring changes in a single fluorophore bound to the enzyme.

US Patent Application No. 2006/078937 discloses a method for sequencing a molecular sequence which comprises supplying an unknown sequence of nucleotides to a single-molecule sequencer comprising a polymerase having a fluorescent donor covalently attached thereto and monomers for the polymerase; exciting the fluorescent donor and detecting emitted fluorescent light.

US Patent Application No. 2006/292583 describes a method of sequencing a nucleic acid which comprises attaching a polymerase to a substrate; allowing a sample nucleic acid and an annealed oligonucleotide to bind to the polymerase in the presence of nucleotides for incorporation into a complementary nucleic acid, wherein the polymerase and nucleotides are cooperatively labelled with donor and acceptor fluorophore that emit a unique signal when a particular nucleotide is incorporated into the complementary nucleic acid and detecting a sequential series of the unique signals as the nucleotides are sequentially added to the complementary nucleic acid.

US Patent Application No. 2007/0172867 to Visigen states that the sequencing system described therein involves a fluorescent tag on the polymerase and unique quenchers on the dNTPs, where the quenchers preferably have distinguishable quenching efficiencies for the polymerase tag. Consequently, the identity of each incoming quencher tagged dNTP is determined by its unique quenching efficiency of the emission of the polymerase fluorescent tag. The signals produced during incorporation are detected and analyzed to determine each base incorporated, the sequence of which generates the DNA base sequence.

However, the disclosure in U.S. Pat. No. '867 does not clarify how the unique quenchers leading to distinguishable quenching efficiencies will be chosen; it is equally unclear how the quenching efficiencies will be measured. Even if these two issues are addressed, the approach based on singly labelled polymerase is confounded by the fluctuations of the fluorescence intensity signal of a single fluorophore occurring due to changes in its photophysical state and/or conformational state. It is well established in the single-molecule fluorescence field that fluorophores enter “dark” states during which they do not emit fluorescence or during which they do not absorb photons (and hence does not emit fluorescence); these states can last for microseconds (e.g., triplet states) up to several seconds. This intermittent emission creates two important problems for all polymerase-based approaches. First, absence of fluorescence emission from a single donor fluorophore (for dark-quencher approaches) will result in loss of information during reading of several base pairs, generating gaps in the sequence to be read, therefore rendering the final sequence inaccurate and unreliable. Second, millisecond-timescale fluctuations will result in a time-average fluorescence intensity, which may lead to an incorrect assignment of a base, since the change in fluorescence intensity will not solely reflect the presence of a specific quencher but also the occurrence of a nucleotide-independent fluorescence-intensity perturbation. The approach that employs dual labelled RNAP and dark-quencher NTPs circumvents the problem by assigning bases only to the events that cause simultaneous and correlated fluorescence-intensity changes (i.e., a significant signal decrease followed by an increase that returns intensity to the initial levels) on both fluorophores.

Furthermore, the Visigen patent application describes a composition comprising a polymerase including at least one pair of molecular tags located at or near, associated with or covalently bonded to a site of the polymerase, where a detectable property of at least one of the tags undergoes a change before, during and/or after monomer incorporation.

This general claim is attempting to cover the use of multiply tagged polymerases for sequencing. The subsequent claims focus on the use of a pair of complementary (primarily fluorescent) probes that report conformational states and conformational changes during monomer incorporation; the changes (presumably) are associated with the binding of a specific nucleotide and may either result in base assignment, or increase the accuracy of base assignment. Again, it is not clear how these fluorescence properties will be translated into sequence information. Moreover, no combination of use of doubly labelled polymerases with dark quenchers is described.

### STATEMENTS OF THE INVENTION

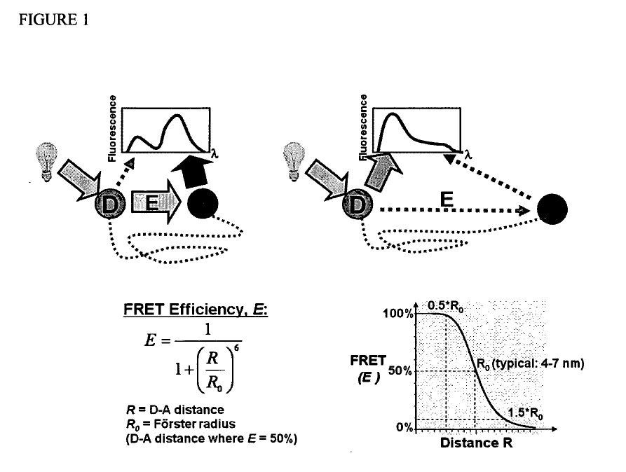

Therefore, according to a first aspect of the invention we provide an assay method for determining the base sequence in a nucleic acid sample which comprises detection of the single base using at least one fluorophore, a quencher group and a nucleic acid polymerase; detecting and deducing the quenching efficiency of the at least one fluorophore. The quenching process, central to the concept of the assay, is based on fluorescence resonance energy transfer (FRET; FIG. 1).

Preferably the quencher group is a dark quencher group.

Preferably the polymerase is modified to include the at least one fluorophore. The fluorophore is located at or near a site on the polymerase where the fluorophore undergoes a fluorescent change upon binding of a quencher (e.g. dark quencher) group (a nucleotide labeled with a quencher (e.g. dark quencher) moiety). For example the fluorescent property (e.g. intensity of fluorescence light emitted by the fluorophore) of the at least one fluorophore has a first value before the fluorophore interacts with a quencher and a second value when interacting with the quencher.

Preferably the polymerase in modified to include multiple fluorophores, for example a pair of fluorophores. The present invention provides a polymerase modified to include at least a pair of fluorophores wherein the fluorophore pair interacts to form a first fluorescence emission profile prior to incorporation of a quencher and a second emission profile following incorporation of a quencher.

Typically one of the fluorophore pair is a fluorophore excited by green light (in the range of 514-560 nm, preferably at 532-nm) and the other is a fluorophore excited by red light (in the range of 633-647 nm, preferably at 638-nm); we define these two fluorophores as “green” and “red” fluorophore, respectively. Fluorophores excited at the blue and infra-red region of the spectrum are also compatible with the assays. The fluorescent properties of the fluorophore pair before, during and/after incorporation of one, or a series, of quencher groups, is converted into an identity of one or more nucleotide, or a sequence of nucleotides, complementary to a sequence of nucleotides in the nucleic acid sample.

Preferably, the assay utilises two fluorophores. The fluorophores should be compatible with single-molecule fluorescence detection; this means that the fluorophore (or fluorescent system, if it is comprised by more than one fluorescent moiety) should be photostable and bright; such fluorophores are typically excited by excitation sources at the visible range (400-800 nm, e.g. 400-700 nm), although fluorophores excitable using near-infrared wavelengths (700 nm-350 μm, e.g. 800 nm-1.2 μm) may also be useful.

Fluorophores that can be used in this platform may be selected from (but not limited to) the group consisting of 5-carboxyfluorescein (FAM), tetramethylrhodamine (TMR), Alexa-Fluor fluorophores (such as Alexa488, Alexa532, Alexa546, Alexa555, Alexa568, Alexa594, and Alexa647; available from Molecular Probes/Invitrogen), BODIPY dyes, ATTO dyes (such as ATTO532, ATTO568, ATTO594, and ATTO647n; available from Atto-tec), cyanine dyes (such as Cy2, Cy3, Cy3B, Cy3.5, Cy5, Cy5.5, and Cy7; available from GE Healthcare) and quantum dots. Although the number of fluorophores may vary, in a preferred aspect of the present invention we provide an assay consisting of 1 fluorophore (a single colour approach) or 2 fluorophores (a dual colour approach), although it is within the scope of the present invention for a plurality of fluorophores/colours to be utilised. A preferable pair of fluorophores may comprise fluorophores Cy3B and ATTO647n.

In contrast to the Visigen patent application, the invention described herein does not rely on the interaction or complementarity between the partners of the pair of fluorescent probes placed on the polymerase (e.g., no FRET is required between the two probes), and does not rely on the presence of a conformational change during monomer incorporation. However, the present invention is compatible with the presence of FRET between the probes placed on the polymerase, provided that the existing FRET is considered as a parameter in the analysis of the time records that result in base sequence assignment. Moreover, the present invention clearly describes how monomer identity can be assigned on the basis of differential quenching of two spectrally distinct fluorescent probes that are incorporated on a nucleic acid polymerase such as T7 RNA polymerase or a DNA polymerase.

The quencher group is a modified nucleotide in particular a nucleotide that is labelled with a quencher moiety. Preferably the quencher group is a dark quencher group. The dark quencher group is a modified nucleotide in particular a nucleotide that is labelled with a dark quencher moiety. As used herein a “dark quencher moiety” is a chromophore that quenches the fluorescence of the at least one fluorophore without emitting light.

A he dark quencher moiety is a chromophore with strong absorbance in the visible region of the electromagnetic radiation spectrum. As such, the dark quencher is able to reduce the fluorescence intensity and fluorescence lifetime of one or more fluorophore(s) by acting as fluorescence resonance energy transfer (FRET) acceptors; importantly, the dark quenchers do not fluoresce either upon direct excitation or upon FRET-based excitation. The dark quencher moiety is attached to a nucleotide, mainly nucleoside triphosphate (NTP) or deoxynucleoside triphosphate (dNTP). The dark quencher will be able to quench the fluorescence of a single fluorophore (for the single colour approach) and of 2 fluorophores (for the dual colour approach). It is preferable that the quencher moiety is covalently bound to the gamma phosphate group of the nucleoside or the beta phosphate group of the nucleoside. It is preferable that the quencher moieties on each nucleotide type are different for example the quencher moiety on A, C, G, T/U are different. The dark quencher may vary, but may be selected from (but not limited to) the group consisting of DABCYL, BHQ1, BHQ2, QSY7, QSY9, QSY21, QSY35, BHQ0, BHQ1, BHQ2, QXL680, ATTO540Q, ATTO580Q, ATTO612Q, DYQ660 and DYQ661; the last five quenchers are commercially available as gamma-phosphate derivatives of nucleotides (from Jena Biosciences). In an especially preferred aspect of the present invention in the assay of the invention at least four quencher groups are present. Preferably each of the at least four quencher groups is different. For example each of the quencher groups includes a quencher moiety and a nucleotide which is different to the quencher moiety and nucleotide of the other quencher groups. Preferably the set of quencher groups used in the assay of the invention includes a quencher group for each of the nucleotides A, G, C and T/U.

Although a variety of nucleotide moieties may be used, the nucleotide moiety may be selected from the group consisting of adenosine triphosphate, cytosine triphosphate, guanosine triphosphate, uridine triphosphate, deoxyadenosine triphosphate, deoxycytosine triphosphate, deoxyguanosine triphosphate and deoxythymidine triphosphate. The assay is also compatible with any nucleic-acid-polymerase-recognised moiety that can be incorporated in a nascent nucleic acid and can report on the identity of the templated nucleotide.

Preferentially, the base is an amine base in a DNA or RNA. Therefore, the polymerase may be selected from the group consisting of a DNA polymerase, an RNA polymerase and a reverse transcriptase. Preferably the polymerase is a RNA polymerase. Preferably still the polymerase is a DNA polymerase.

In a further aspect of the invention there is provided a method for determining the nucleic acid sequence of a nucleic acid sample the method comprising the steps of:
i) providing a composition comprising a nucleic acid sample, for example a DNA sample, and a modified polymerase wherein the modified polymerase includes a first fluorophore and a second fluorophore;
ii) contacting the composition in (i) with at least one quencher (e.g. dark quencher) group; and
iii) detecting changes in the fluorescent properties (e.g. detecting and deducing the quenching efficiency) of the first and second fluorophore before, during and/or after step (ii); and
iv) optionally converting the information in (iii) into an identity of one, or a plurality of, nucleotide(s) complementary to a sequence of nucleotides in the nucleic acid sample.

In a preferred method of the invention the method comprises the step of exciting the fluorophores with an excitation source.

Changes in the fluorescent properties of the fluorophores may include the duration of fluorescence, intensity of fluorescence and/or frequency of fluorescence.

In a preferred method of the invention at least four quencher groups are present. Preferably each of the at least four quencher groups is different. For example each of the quencher groups includes a quencher moiety and a nucleotide which is different to the quencher moiety and nucleotide of the other quencher groups. Preferably the set of quencher groups used in the assay of the invention includes a quencher group for each of the nucleotides A, G, C and T/U.

According to a further aspect of the invention we provide a nucleotide triphosphate (NTP) probe, the NTP probe consisting of an NTP having a fluorophore moiety attached thereto; and a quencher moiety sufficiently proximal to the fluorophore to prevent or hinder fluorescence of the fluorophore; preferably the NTP probe comprises at least 4 proximal quenching moieties as hereinbefore described.

According to a yet further aspect of the invention we provide a kit comprising one or more fluorophore moieties and attached NTPs; a plurality, e.g. four, associated quenchers and at least one polymerase.

In a yet further aspect of the invention there is provided a modified polymerase, such as a modified RNA polymerase, wherein the modified polymerase comprises a polymerase which has been modified to include multiple fluorophores, for example a pair of fluorophores, such as defined herein.

In a further aspect the inventions provides the use of a modified polymerase, such as an RNA polymerase in nucleic acid (e.g. DNA) sequencing.

In a further aspect of the invention there is provided a kit comprising a modified polymerase and at least one quencher (e.g. dark quencher) group. Preferably the kit comprises a set of quencher (e.g. dark quencher) groups wherein the set comprises at least 4 quencher groups (e.g. dark quencher groups such as defined herein).

### DETAILED DESCRIPTION OF THE INVENTION

As discussed, the platform is compatible with various nucleic acid polymerases. An embodiment using RNAP will be described in detail. A typical procedure for RNAP-based single-molecule DNA sequencing comprises the following steps:
1) Immobilisation of modified genomic, amplified or synthetic DNA on a solid support
2) Introduction of a fluorescently labelled RNAP and binding of RNAP to promoter DNA
3) Introduction of quencher-modified nucleotides
4) Initiation of RNA synthesis and subsequent extension of RNA during which the DNA sequence is read by monitoring fluorescence intensity levels

As described, implementation of the procedure requires modified transcription components. A typical set of reagents will include:
1) Genomic DNA modified to incorporate a promoter and an immobilisation tag
2) A fluorescently labelled RNAP
3) 4 quencher-modified NTPs

### DNA Preparation and Surface-Immobilisation

To prepare the modified genomic DNA, the genomic DNA will be ligated to a short (e.g. 30-50 base pairs) double-stranded DNA sequence that contains a strong promoter sequence for the RNAP to be used; such protocols use commercial reagents and are routine procedures. In addition, the added short DNA will contain a surface-immobilisation tag (e.g., a biotin moiety) that anchors the DNA to a modified solid support. The solid support (e.g., quartz or glass) has been modified to prevent non-specific adsorption of biomolecules (polymerases, NTPs, DNA); this is achieved using published methods that employ hydrophilic polymers (such as poly-ethylene-glycol, PEG) combined with low ratios (˜1%) of modified hydrophilic polymers (such as biotin-PEG) which allow specific immobilisation of modified biomolecules. In the preferred case that involves the use of biotin-PEG, addition of a layer of streptavidin or neutravidin (a protein that binds very tightly to biotin) provides a point for specific attachment of biotinylated DNA fragments or biotinylated polymerases. This strategy allows stable and specific immobilisation of the modified genomic DNA on the surface and enables the imaging of fluorescence using wide-field excitation and imaging of the emitted fluorescence.

In another version of the assay, the fluorescent RNAP is immobilised on the solid support (e.g., using the biotin-streptavidin interaction or the hexahistidine-tag-Ni²⁺:nitriloacetic acid interaction) and the genomic DNA is followed to allow formation of RNAP-DNA interactions, initiation of transcription and processive extension of the RNA; the latter process is the one resulting in reading the DNA sequence.

### Fluorescent Labelling of Nucleic Acid Polymerases

To prepare the fluorescently labelled RNAP, one or more specific reactive sites are generated on an RNAP; these sites can then be reacted with a specific reactive form of one or more fluorophore(s) to yield the labelled RNAP derivatives. To prepare a singly or doubly labeled RNAP, RNAP derivatives without surface accessible cysteines are generated using site-directed mutagenesis, a standard molecular-biological technique. For many polymerases (including T7 RNAP, Klentaq DNAP and the Klenow fragment of DNAP I), such derivatives are already available. As a specific example, T7 RNAP has been modified to remove 7 of its 12 native cysteine residues; this protein derivative (“Cys-light T7 RNAP”) has essentially no surface-exposed cysteines.

The polymerase derivatives with no surface-exposed cysteines are modified (using site-directed mutagenesis) to yield polymerase derivatives with one or more surface-exposed cysteines. The cysteine substitution sites for the mutagenesis are chosen considering the distances of the fluorophores from the nucleotide binding site and ensuring that the original amino acid (before the mutation to cysteine) is not conserved or does not perturb the polymerase folding or perturb its polymerizing activity in a significant way. As a specific example, availability of a published crystal structure for the T7 RNAP elongation complex in the presence of an incoming nucleotide (1s0v) allow precise selection of the sites to be labelled to maximise the signal of the fluorescence-based assay.

To generate a singly labelled RNAP, a single surface-exposed cysteine derivative of an RNAP is reacted with a maleimide (or iodoacetamide) derivative of a fluorophore. This approach has been used to generate multiple protein derivatives with a single surface-exposed cysteine introduced at multiple sites of interest. High homology of T7 RNAP with other bacteriophage RNAPs (e.g., SP6 and T3 RNAPs) allows similar procedures to be used with a wide range of single-subunit RNAPs.

Once multiple surface-exposed cysteines are introduced at sites of interest, they are labeled through reactions with maleimide (or iodoacetamide) derivatives of commercially available fluorophores to prepare multiply labeled polymerases. For example, for a doubly labelled RNAP, two surface-exposed cysteines can be labelled with a “green” (G) and “red” (R) fluorophore. If the reactivity of the two cysteines is substantially different, then the use of an equimolar amount (or small molar excess, in the order of 50-100% of the first reactive fluorophore over protein) will primarily label the most reactive site; after the first reaction is complete (i.e., after an incubation of >2 hrs), subsequent addition of the second reactive fluorophore will react with the least reactive site and yield a site-specifically doubly labeled polymerase; free and unreacted fluorophore are quenched using an excess of a thiol-containing reagent (e.g., dithiothreitol or beta-mercaptoethanol) and are removed using standard methods such as gel filtration or dialysis. Such procedures have been used for labeling the Klenow fragment of DNAP I.

A DNA polymerase labelled with dual fluorophores is described in Allen et al., Protein Sci. 2008 March; 17(3):401-8.

Other methods for introducing sites in protein for site-specific labelling can also be used, such as use of the CCPGCC motif for labelling with bi-arsenical fluorescence reagents and introduction of (His)_{n>5} (poly-histidine) motif for labeling with nickel-nitrilotriacetic acid fluorescence reagents. Multi-subunit RNAPs, such as the Escherichia coli RNAP can also be used for the assay provided that the fluorophores are site-specifically incorporated in the core RNAP machinery that is present throughout the transcription pathway.

The labelling can also be statistical, leading to GG-, GR-, RG-, and RR-labelled species which can be identified by the signal intensities at the single-molecule level; in the “GR” protein derivative, the first cysteine (cysteine-1) is labeled with the green fluorophore whereas the second cysteine (cysteine-2) is labeled with the red fluorophore; in the “RG” protein derivative, the labeling sites are reversed. Such procedures have been reported for labeling Klentaq DNAP I.

### Preparation of Quencher-Modified Nucleotides

In the case of the RNAP-based approach, we use commercially available quencher-modified nucleotides (from Jena Biosciences). In the case that the dark-quencher is not commercially available, quencher-nucleotide derivatives can be prepared by incubating reactive-forms of nucleoside triphosphates (NTPs) with reactive forms of several distinct dark quenchers. The quenchers will be introduced to the gamma-phosphate of the NTPs, and as such, will not accumulate during the transcription reaction. Instead, the dark quenchers will lead to the onset of fluorescence quenching that starts upon their binding to the polymerase-template complex and will cease upon the dissociation of the pyrophosphate product of the nucleic-acid extension reaction. The modification of the gamma-phosphate with chromophores has been described before and its chemistry is straightforward. Quenchers can also be introduced in the beta-phosphate group of nucleotides since the beta-phosphate is included in the part of the nucleotide that is cleaved during each cycle of nucleic-acid extension (see U.S. Pat. No. 6,399,335 for several synthetic routes of the foregoing).

### Imaging Single Transcription Complexes.

Immobilisation of modified genomic DNA on a glass (or quartz) surface allows addition of labelled RNAPs and labelled nucleotides for the formation of RNAP-DNA complexes and initiation of transcription. In the case of large DNA fragments, methods that allow extension of long DNA fragments (e.g., use of flow, microfluidic channels or molecular combing) will permit monitoring of the activity of several RNAPs on a single DNA fragment.

Upon binding of a fluorescent RNAP on surface-immobilised DNA, single RNAP molecules can be imaged using wide-field imaging approaches, with total internal reflection fluorescence (TIRF) microscopy being preferable. TIRF microscopy uses evanescent-wave excitation within a thin layer off a surface and wide-field imaging on an ultrasensitive camera (e.g., iXon+897, an electron-multiplying CCD camera from Andor Technology) to observe surface-immobilised molecules for extended period. When a light beam crosses an interface into a medium with a lower index of refraction at an angle larger than a critical angle, an evanescent wave is generated close to the interface, with the intensity of the field I(d) decaying exponentially with distance: I(d)=I₀e^{-z/d} where z is the distance from the surface and d is a characteristic depth, typically on the order of 100 nm. Because the evanescent wave decays rapidly with distance, single molecules immobilised on the surface can be imaged on the camera with little background from molecules diffusing in solution at focal planes away from the surface. Several ways exist for generating evanescent waves for TIRF microscopy; the preferred methods are objective-type TIRF and prism-type TIRF. Objective-type TIRF uses high numerical aperture oil-immersion objectives (NA≧1.4, 60× or 100× magnification and ultra-low fluorescence background) to generate a parallel narrow beam that reaches the glass-water interface at the required critical angle; the fluorescence is collected through the same objective. Prism-type TIRF uses a prism in optical contact with a quartz slide to excite fluorescence molecules at the bottom of the slide; the fluorescence is collected through a water-immersion objective (NA of 1.2, 60-100× magnification; with long working distance) distal to the prism and the excitation source. Simultaneous as well as alternating excitation of a “green” and “red” fluorophores using TIRF-based imaging is achieved using simultaneous or alternating-laser excitation by two lasers each of which primarily excites one of the two fluorophores.

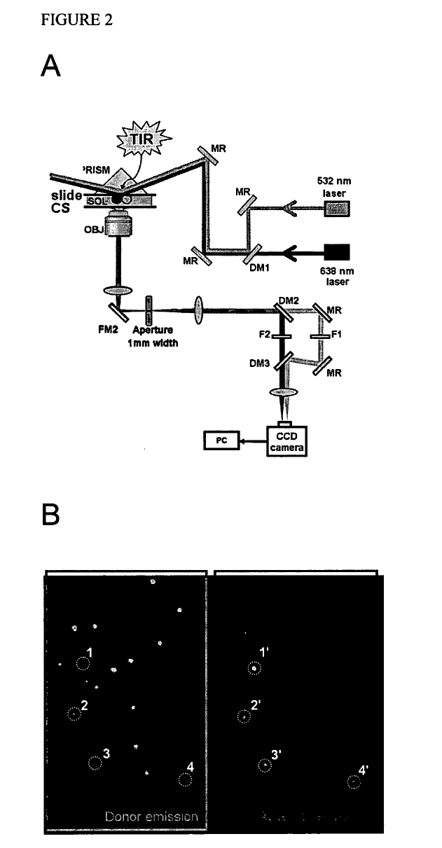

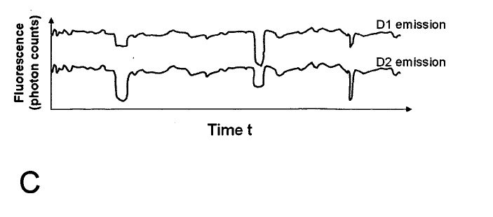

In all TIRF approaches, immobilised molecules appear as spots of fluorescence intensity over a dark background (FIG. 2B). It is important to control the concentration of PEG-biotin and of biotinylated DNA to ensure sparse coverage of the surface by labelled complexes. Depending on the magnification of the objective used and the number of cameras for imaging, up to 1000 fluorescent molecules can be imaged in a single frame. Using the objective-type TIRF, areas of 30×30 μm are typically monitored; prism-type approaches can illuminate larger areas (100×100 μm) at the expense of complexity in sample and imaging-chamber handling.

Monitoring the fluorescence intensity in two channels over time allows one to monitor transient quenching of the green and red fluorescence, and to use this information to assign bases. Since the amount of information obtained from a single-molecule is proportional to the time that a fluorophore emits before it eventually photobleaches due to irreversible photochemical reactions, we use measurement conditions (buffer compositions, oxygen exclusion, excitation power) that maximise the duration of observation by minimising photobleaching. To extend the length of the time traces, we perform the observation in enclosed chambers filled with observation buffers that contain an enzymatic oxygen scavenging system (containing 1665 units glucose oxidase, 26,000 units catalase and 1% glucose) and a triplet-state scavenger (TROLOX, ˜2 mM). Using these conditions, we have routinely obtained single-molecule fluorescence traces that last longer than 15 min; such extended photobleaching lifetimes should allow continuous monitoring of thousands of nucleotide incorporation events per single fluorescent RNAP molecule. The in vitro transcription rate for T7 RNAP is ˜100 nt/sec, therefore in principle, 60,000 nucleotides can be read in 10 min from a single RNAP molecule. Our current instrumentation allows us to monitor green and red fluorescence with signal-to-noise ratios of >5 for exposure time of 4 ms, which should allow us to monitor reliably multi-step reactions such as the nucleotide addition reaction by RNAP. If necessary, the transcription rate by T7 RNAP can be decreased by reducing the concentration of NTPs during the reaction.

### Data Analysis

Movies of fields of views for two different spectral regions (typically, the main emission bands of the “green” and “red” fluorophore) are recorded using software provided by the camera manufacturer. The movies are then analysed to achieve proper image registration, automated particle detection and association, and fluorescence photometry. Proper image registration is achieved using a calibration performed using measurements on 0.2 μm fluorescence beads or on microfabricated patterns). Automated particle detection and association is performed by identifying particles in each detection channel using sequential spatial low- and high-pass filters, followed by a nearest-neighbour search in the two channels. Fluorescence photometry is performed on the identified particles by calculating the total fluorescence intensity of a small circle centred at the particle (aperture photometry), corrected for background photons. In a different method, we use fitting of two-dimensional Gaussian functions to the intensity profile to the particles and obtain their total intensity from the Gaussian function (profile-fitting photometry).

### Sensing Nucleotide Identity Using FRET

The single-colour approach relies on differential quenching of a fluorophore on an RNAP by a set of quencher-modified NTPs. Each different nucleotide is labelled at the gamma-phosphate with a distinct dark quencher. The quenchers reduce the fluorescence of the RNAP fluorophore through fluorescence resonance energy transfer (FRET).

FRET usually serves as a proximity-based assay, since the efficiency E of FRET depends on the distance between two fluorophores, a donor and an acceptor (FIG. 1). According to the Förster theory, FRET efficiency E is related to the distance between the fluorophores by

$$E = \frac{1}{1 + {( R / {R}_{0} )}^{6}} \qquad ( eq . 1 )$$

Where R₀ represents the Förster radius of the pair of fluorophores used:

$${R}_{0}^{6} = \frac{9000 ( ln 10 ) {κ}^{2} {Q}_{D}}{128 {π}^{5} {Nn}^{4}} {\int }_{0}^{∞} {F}_{D} ( λ ) {ɛ}_{A} ( λ ) {λ}^{4} d λ \\ with \\ {κ}^{2} = {( cos {θ}_{T} - 3 cos {θ}_{D} cos {θ}_{A} )}^{2} \qquad ( eq . 2 )$$

The Förster radius Rₒ for a specific donor-acceptor FRET pair is the distance at which FRET efficiency is 50%. The Rₒ value is a measure of the dynamic scale of the FRET measurement and is specifically related to the donor and acceptor fluorophore; it can be determined experimentally by measuring the quantum yield of the donor Q_{D}, the fluorescence spectrum of the donor F_{D}(λ), and the wavelength-dependent extinction coefficient of the acceptor ε_{A}(λ), the refractive index of the medium n (N: Avogadro's number). The relative orientation of the dye is expressed by κ², derived from the angle between the dipole moment of the donor and acceptor with respect to the connecting line (θ_{D} and θ_{A}) and relative to each other (θ_{T}). In case of freely rotating fluorophores, κ² can be approximated to ⅔.

Since the Rₒ is proportional to the spectral overlap of donor emission and acceptor absorption spectra (summarized by the integral in equation 2), the process of FRET is entirely compatible with acceptors that do not emit fluorescence; the only requirement for an FRET acceptor is to possess absorption spectra with significant overlap with donor emission spectra. A chromophore that acts a FRET acceptor but does not emit fluorescence is usually referred to as a “dark quencher”. Since the dark quencher does not produce any FRET-sensitized emission, the FRET process is monitored by measuring the reduced quantum yield of the donor (a fluorescence-intensity-based measurement) or the reduced fluorophore lifetime of the donor fluorescence. In this approach, we will measure the FRET efficiency by monitoring the reduced quantum yield as reduced fluorescence intensity of single transcription complexes (FIG. 2C).

### Approach Using Singly Labelled RNAP

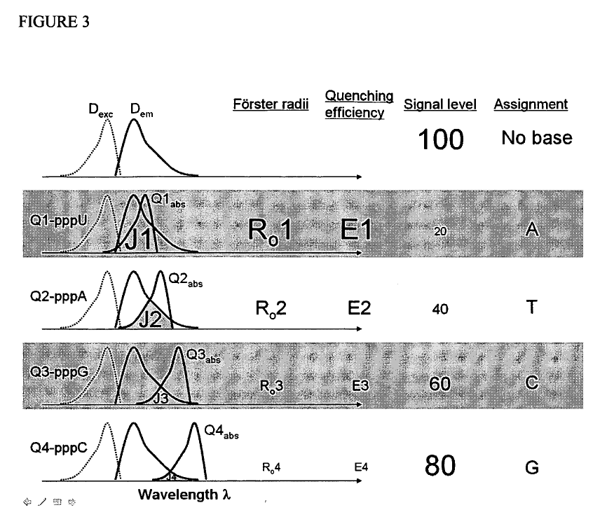

Since the single-colour approach uses a single site on RNAP, the distance between the fluorophore and each dark quencher will be similar for all quencher-modified NTPs. To distinguish between different quenchers (and hence different nucleotides), one must choose quenchers with different characteristic distance Rₒ relative to the fluorophore on RNAP). This can be achieved by choosing dark quenchers with absorption spectra that result in different spectral overlap with the donor emission spectrum (FIG. 3). This will allow different FRET efficiencies for different quenchers at a similar distance from the RNAP donor fluorophore.

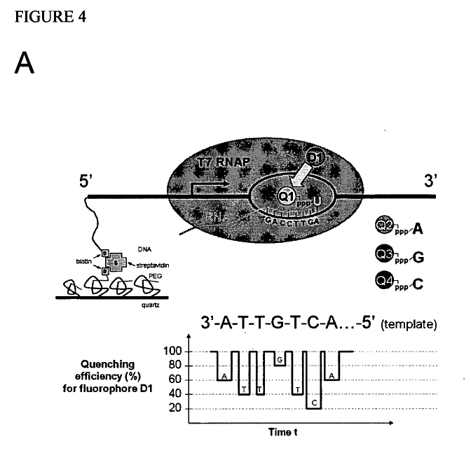

In the context of transcription by RNAP, each NTP binding and incorporation is observed as a short dwell at one of 4 levels of fluorescence quenching (FIG. 4A). Each dwell at a quenched state ends by returning to an unquenched state; the unquenched state lasts from the release of the quencher-pyrophosphate group to the binding of the next NTP to the active site. Base assignment is done on the basis of the fluorescence intensity during a single incorporation cycle. Since the quenchers are not fluorescent, this approach should allow polymerase-based sequencing at μM concentrations without the excitation volume confinement implemented using zero-mode waveguide (ZMW) technology or micro-fabricated nanochannels (note: these technologies allow operation of DNA polymerases at μM NTP concentrations, ensuring polymerase processivity without unacceptably high fluorescence background from the high concentration of free NTPs). The ability to operate without ZMW simplifies greatly the preparation of the substrates and will have favourable cost considerations.

### Approach Using Doubly Labelled RNAP

A more robust way to assign bases uses a dual-fluorophore approach. The dual-colour approach (and in principle n-colour approach) relies on differential quenching of two spectrally distinct fluorophores on an RNAP by a set of 4 quencher-modified NTPs (FIG. 4B). This approach circumvents one of the potential complications which can confound single-molecule DNA sequencing approaches based on single-molecule fluorescence spectroscopy: the fluctuations of fluorescence intensity of a single fluorophore due to changes in its photophysical state and/or conformational state. It is well established in the single-molecule field that fluorophores enter “dark” states during which they do not emit fluorescence or absorb photons; these states can last for microseconds (e.g., triplet states) to several seconds; moreover, fluorophores can undergo spectral shifts that reduce or increase their fluorescence intensity. These photophysical phenomena can result in two main problems for all polymerase-based approaches. First, absence of fluorescence emission from a single donor fluorophore (for dark-quencher approaches) or a single-acceptor fluorophore (for bright-acceptor approaches) will result in loss of information during reading of several base pairs, generating gaps in the sequence to be read. Second, millisecond-timescale fluctuations will result in a time-average fluorescence intensity, which may lead to an incorrect assignment of a base, since the change in fluorescence intensity will not solely reflect the presence of a specific quencher but also the occurrence of an NTP-independent fluorescence-intensity perturbation.

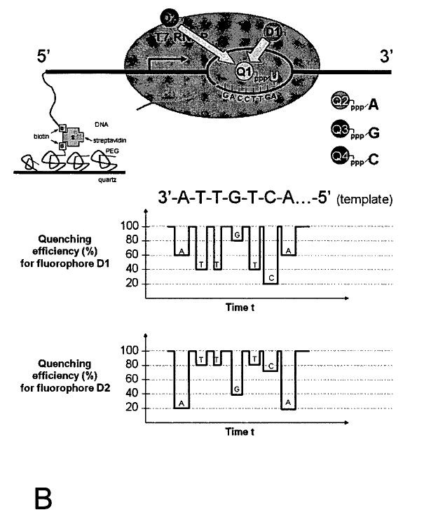

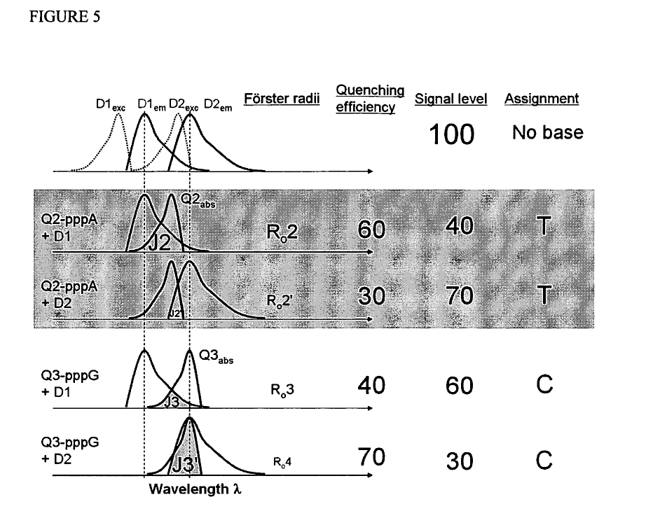

The principle of dual-colour assay is illustrated in FIGS. 4B, 5 and 6. In the case of transcriptional sequencing, RNAP is labelled with two fluorophores (D1 and D2 in FIG. 5) with emission spectra with maxima spaced by ˜100 nm. The fluorophores are separated by a large distance (>8 nm) that ensures that FRET between fluorophores D1 and D2 is negligible (FRET efficiency less than 10%). We note that in case the fluorophores are within range for significant FRET, the existence of FRET does not preclude the assignment base on binding of dark-quencher NTP since it will still result in a correlated quenching of the fluorophores on the polymerase to distinct intensity levels that allow base assignment and distinguish these signal changes from changes that arise due to changes in the donor or acceptor photo physical state. Upon binding of quencher-labelled ATP (which has been selected to cause differential quenching of the two fluorophores), fluorophore D1 is quenched by 60%, whereas fluorophore D2 is quenched by 30%.

There are two major advantages with this labelling scheme. First, ATP binding initiates simultaneous quenching of the D1 and D2 fluorescence, and the release of the quencher-bound pyrophosphate leads to simultaneous de-quenching of the D1 and D2 fluorescence; this correlated change in signal for the two fluorophores clearly distinguishes the NTP-based dwell time in the quenched state from photophysical changes in the two fluorophores since the latter changes will not be correlated (in the absence of FRET between fluorophores D1 and D2).

Second, each NTP is associated not with one but with two levels of quenching efficiency, increasing the accuracy of the assay and therefore of the read DNA sequence. To increase the accuracy of the assay, quenchers specific to the rest of the nucleotides can be chosen to maximise the difference between the levels of quenching between the 4 nucleotides. For example, upon binding of quencher-labelled GTP (which has been selected to cause differential quenching of the two fluorophores but to a degree substantially different to ATP), fluorophore D1 is quenched by 40%, whereas fluorophore D2 is quenched by 60%. The difference in quenching levels between nucleotides can be summarised in a ratiometric expression referred to as relative probe stoichiometry, S:

$$S = \frac{{F}_{D 1}}{{F}_{D 1} + {F}_{D 2}} \qquad ( eq . 3 )$$

where F_{D1} and F_{D2} are the fluorescence intensities of the fluorophore D1 and D2 either in the presence or absence of quenching, respectively. This simple ratio essentially reports on whether fluorophore D1 is quenched more or quenched less by a quencher-labelled NTP compared to fluorophore D2. In the absence of quenching, the laser-excitation power is adjusted to obtain an S˜0.5 in the absence of quenching.

Moreover, the extent of quenching of both fluorophores can also be used for discrimination between dark quenchers (and, therefore, between bases). The extent of quenching can be summarised in the following simple ratiometric expression:

$$\begin{matrix}{Q}_{dark , D 1} = \frac{{F}_{D 1}^{Q}}{{F}_{D 1}} & ( eq . 4 ) \\ {Q}_{dark , D 2} = \frac{{F}_{D 2}^{Q}}{{F}_{D 2}} & ( eq . 5 ) \\ {Q}_{dark} = \frac{{F}_{D 1}^{Q} + {F}_{D 2}^{Q}}{{F}_{D 1} + {F}_{D 2}} & ( eq . 6 )\end{matrix}$$

where F_{D1} and F_{D2} are the fluorescence intensities of the fluorophore D1 and D2, respectively in the absence of quenching, and F_{D1}^{Q} and F_{D2}^{Q} are the fluorescence intensities of the fluorophore D1 and D2, respectively, in the presence of quenching. These expressions, combined with the S ratio, will lead to the assignment of the correct base.

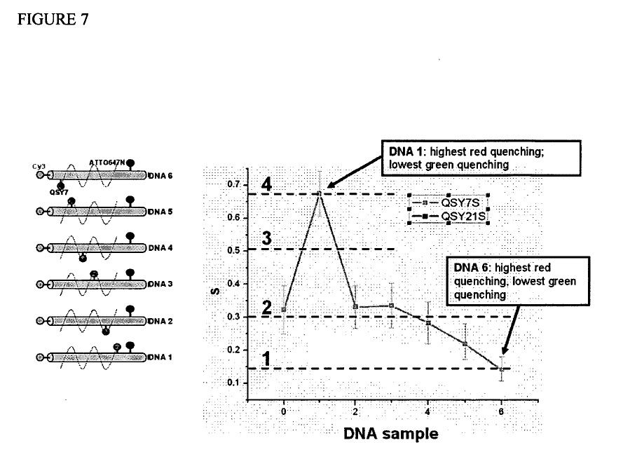

An example of different stoichiometry values arising from different FRET efficiency for two fluorophore-dark quencher pairs on the same DNA molecules is shown in FIG. 7. The results are obtained by observing single diffusing DNA molecules. The FRET efficiency changes by moving the site of incorporation of a dark quencher (QSY7) from the end of a double-stranded DNA proximal to a red green fluorophore (ATTO647N) to the end of the DNA proximal to a green fluorophore (Cy3). As the fluorophore moves from a site proximal to the red fluorophore (as in DNA1) to the site proximal to the green fluorophore (as in DNA6), the stoichiometry ratio S decreases from a high value of ˜0.7 to a low value of ˜0.15, sampling several distinct levels at the intermediate positions. The sampled levels of S demonstrate the feasibility of generating multiple levels of stoichiometry within the 0.15-0.7 range. Careful selection of labelling sites and selection of dark-quencher sets should allow generation of 4 distinct stoichiometry levels (0.15, ˜0.3, ˜0.5 and 0.7) for single molecules labelled with the fluorophore combination of Cy3/ATTO647N. Additional sensitivity is expected for the preferred fluorophore combination of Cy3B/ATTO647N (due to the higher photon count of Cy3B over Cy3). The unquenched state can be distinguished not only on the basis of S, but mainly on the basis of quenching efficiencies.

### Example of an Application Using T7 RNAP as the Sequencing Polymerase

An example of an application to the use of T7 RNAP is described below. The experimental design is based on the preferable use of Cy3B and ATTO647N as the two fluorophores in the approach that uses a doubly labelled RNAP. Their choice is based on the fact that both Cy3B and ATTO647N are bright, photostable and well characterised fluorophores that can be excited by high-power, stable solid-state lasers.

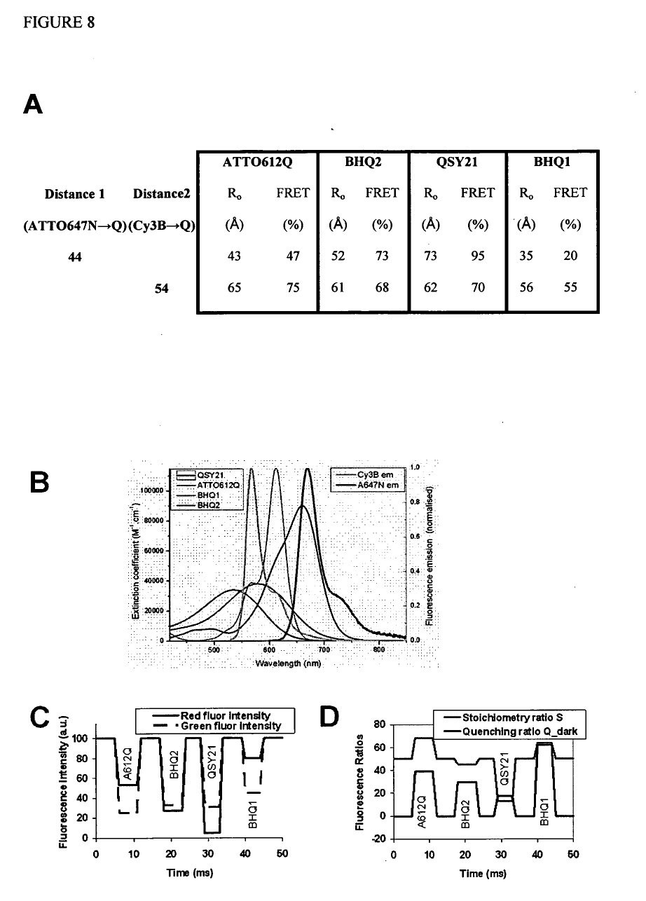

On this basis, we calculated Rₒ values for several fluorophore-dark quencher FRET pairs; the results are summarised in FIG. 8A. Considering the need to generate 4 different sets of stoichiometry and quenching efficiency, we chose 4 dark quenchers (BHQ1, BHQ2, QSY21, ATTO612Q) to be used with our fluorophores of choice. The Rₒ values and the spectra of the fluorophores and dark quenchers are shown in FIGS. 8A and 8B, respectively.

Based on the Rₒ values, it is best to place ATTO647 at ˜45 Å from γ-phosphate and Cy3B at ˜55 Å from γ-phosphate, and in a way that minimises Cy3B→ATTO647N FRET. This dye arrangement allows discrimination of the identity of dark quencher based on quenching efficiencies while it minimises FRET between the fluorophores, since presence of substantial FRET between fluorophores may complicate the data analysis. Several sites on the T7 RNAP elongation complex (as indicated by a structure of the complex in the presence of the incoming nucleotide, 1s0v) that satisfy the criteria exist, giving only a ˜10% FRET between fluorophores. For example, fluorophore D1 can be incorporated in each of several sites on the N-terminal domain, whereas fluorophore D2 can be incorporated in each of several sites on the thumb domain. The presence of low D1→D2 FRET efficiency can be considered in the analysis of the timetraces and factored in the expected stoichiometries and quenching ratios. Importantly, blinking of either the donor or the acceptor will not be mistaken as nucleotide binding and incorporation. The exact values of all FRET efficiencies will depend on the actual distances between chromophores (which depend on the length and configuration of the chromophore linkers), the spectral properties of the chromophores in the protein context (which will be influenced by the local environment) and the rotational freedom of the chromophores (which will influence the orientation factor κ², which in turn affect the value of Rₒ).

With the previous consideration of D1→Q and D2→Q distances and Rₒ values, the expected fluorescence intensities of D1 and D2 due to quenching, as well as the stoichiometry values and quenching ratios, are easily calculated (FIG. 8A, 8C-D). As can be seen by the calculated timetraces of FIGS. 8C and D, the combination of the two fluorescence ratios should clearly distinguish between the four different quenchers, and therefore between the 4 different bases that they represent.

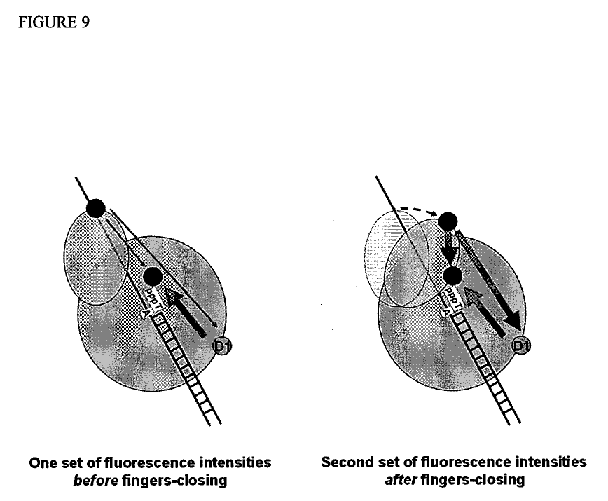

Another embodiment of the dark-quencher based assay takes advantage of conformational changes occurring during the single nucleotide addition cycle; this embodiment will be discussed in the context of DNAPs, for which the conformational changes are better characterised; it is likely that such conformational changes also occur in T7 RNAP, which share several common features with DNA polymerases. Upon addition of the correct dNTP, the fingers' domain of several members of the DNAP I family (e.g., Klenow fragment and Klentaq) assume a closed conformation through a rotational movement that ensures optimal contact of the correct dNTP to the template DNA and to THE NTP binding site on the DNAP. Therefore, if D2 is placed on the fingers' domain (FIG. 9), and D1 is placed along the direction of motion and at distances where there is significant D1→D2 FRET, the system will allow monitoring of the rotational movement. At the same time, D1→Q and D2→Q FRET efficiencies are also significant and distinct for the different dark-quencher pairs. Therefore, the DNAP complex with primer-template DNA (which is presumed to be in the open state) will be characterised by one set of S and Q_{dark} values; binding and incorporation of the correct dNTP system will generate a second set of S and Q_{dark} values. Therefore, provided that there is enough temporal resolution to resolve both conformational states, each dark quencher (and, as a result, each dNTP) is characterised by a set of 4 ratios, a fact that increases the accuracy of the assay.

For a discussion of how the present invention contrasts with closely related prior art, please see the section “Background of the invention”.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The invention will now be illustrated by way of example only and with reference to the accompanying figures:

FIG. 1

The basic principles of fluorescence resonance energy transfer which can act as a molecular ruler for nanoscale distances.

FIG. 2

A. Instrumentation for dual colour prism-based total-internal reflection fluorescence microscopy. This example uses a 532-nm laser that primarily excites one fluorophore on RNAP and a 638-nm laser that primarily excites a second, spectrally distinct fluorophore on RNAP. The beams are combined and directed to the sample where a prism is used to generate, through total-internal reflection (TIR), an evanescent wave on the bottom of a quartz slide; this excited the fluorophores attached to the polymerase. Fluorescence photons are collected through the objective, spectrally separated and are imaged side-by-side on the chip of an ultra-sensitive CCD camera. MR: mirror; DM1, DM2, and DM3: dichroic mirrors; CS: coverslip; OBJ: objective; FM: flipping mirror; F1, F2: optical filters.

B. An image of immobilised molecules emitting in two different spectral regions (green on the left panel; red on the right panel). Spots 1 and 1′ correspond to the green and red emission, respectively, arising from a single immobilised RNA polymerase-DNA complex. Spots 2-4 and 2′-4′ represent another 3 complexes. The intensity of its spot represents the intensity of fluorescence emission from each fluorophore.

C. Schematic of how the fluorescence intensities of the two fluorophores are expected to change with time upon binding and hydrolysis of quencher-modified NTPs.

FIG. 3

Concept of using 4 different quenchers to generate 4 different levels of fluorescence quenching based on the difference in Forster radii between the 4 the different fluorophore-quencher FRET pairs (approach using singly labelled polymerases).

FIG. 4

A. Example of a strategy using singly labelled polymerases and 4 different quenchers to monitor nucleic acid sequence based on quenching levels of fluorophore D1 quenching.

B. Example of a strategy using doubly labelled polymerases and 4 different quenchers to monitor nucleic acid sequence based on quenching levels of fluorophore D1 and fluorophore 2.

FIG. 5

Example of combining the use of quenchers with the use of two fluorophores on a polymerase to increase the confidence of base assignment by implementing differential quenching of the fluorophores by each quencher.

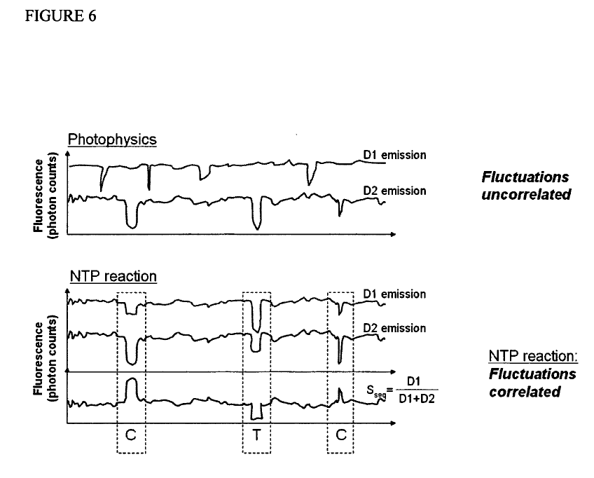

FIG. 6

Schematic of time traces resulting due to photo physical changes (top) and quencher-NTP binding to doubly labelled polymerase (bottom).

FIG. 7

Example of different stoichiometry values arising from different FRET efficiency for two fluorophore-dark quencher pairs on single DNA molecules. Left panel: double-stranded DNA molecules labelled with a green fluorophore (Cy3), a red fluorophore (ATTO647N) and a dark quencher (QSY7) at 6 different positions along the DNA. Right panel: the change in FRET efficiencies for the Cy3→QSY7 energy transfer and the ATTO647N→QSY7 as the QSY7 is moved to a different position on DNA results in large changes in stoichiometry for the populations of single diffusing DNA molecules. The sample was studied using single-molecule FRET microscopy combined with alternating laser excitation. Blue dashed lines: 4 distinguishable levels of stoichiometry that can be generate using combinations of fluorophore with a set of dark quenchers.

FIG. 8

A. Four preferred dark quenchers and their FRET efficiencies for two strategically positioned fluorophores D1 and D2 on the T7 RNAP elongation complex.

B. Emission spectra of fluorophores D1 and D2, and absorbance spectra of the four dark quenchers.

C. Simulated timetrace of D1 and D2 fluorescence intensity as a function of the binding and cleavage of each of the 4 dark-quenchers in panels A and B.

D. Simulated timetrace of stoichiometry ratio S and of quenching efficiency Q_dark as a function of the binding and cleavage of each of the 4 dark-quenchers in panels A and B.

FIG. 9 A schematic of a DNAP-DNA-dNTP complex in the open and complex states along with the two fluorophores (D1 and D2) and a dark quencher in the incoming nucleotide (a dTTP, as dictated by the complementary base on the template). Each state is associated with a set of three significant FRET processes (orange arrows), the measurement of which will increase the accuracy of the assay.

1. An assay method for determining the base sequence in a nucleic acid sample which comprises detection of a single base in said sequence using a polymerase modified to include at least one fluorophore and a quencher group; and detecting and deducing the amount of energy transfer.

2. An assay according to claim 1 wherein the polymerase is modified to include a first fluorophore and a second fluorophore.

3. An assay method according to claim 1 wherein the first and second fluorophore interact to form a first fluorescence emission profile prior to incorporation of the dark quencher and a second emission profile following incorporation of the dark quencher.

4. An assay method according to claim 1 wherein the fluorophore is excited by an excitation source at the visible range (400-700 nm).

5. An assay method according to claim 1 wherein the fluorophore is excited by an excitation source at the infra red range (700 nm-350 μm).

6. An assay method according to claim 1 wherein the fluorophore is selected from the group consisting of 5-carboxyfluorescein (FAM), tetramethylrhodamine (TMR), Alexa-Fluor fluorophores, BODIPY dyes, ATTO dyes, cyanine dyes and quantum dots.

7. An assay method according to claim 2 wherein the first and second fluorophores consist of Cy3B and ATTO647n.

8. An assay according to claim 1 wherein the quencher group is a dark quencher group.

9. An assay method according to claim 8 wherein the dark quencher group is a modified nucleoside triphosphate (NTP) or a dNTP.

10. An assay method according to claim 9 wherein the dark quencher moiety is covalently bound to the gamma- or beta-phosphate group of the NTP.

11. An assay method according to claim 8 wherein the dark quencher is selected from the group consisting of DABCYL, BHQ1, BHQ2, QSY7, QSY9, QSY21, QSY35, ATTO540Q, ATTO580Q, ATTO612Q, DYQ660 and DYQ661.

12. An assay method according to claim 1 wherein the assay comprises at least four quencher groups.

13. An assay method according to claim 12 wherein the assay comprises at least four dark quencher groups.

14. An assay method according to claim 12 wherein each of the at least four quencher groups is different.

15. An assay method according to claim 12 wherein each of the quencher groups includes a quencher moiety and a nucleotide which is different to the quencher moiety and nucleotide of the other quencher groups.

16. An assay method according to claim 9 wherein the NTP is selected from the group consisting of adenosine triphosphate, cytosine triphosphate, guanosine triphosphate, uridine triphosphate, deoxyadenosine triphosphate, deoxycytosine triphosphate, deoxyguanosine triphosphate, deoxythymidine triphosphate, modified DNA, modified RNA or a nucleic acid including DNA or RNA.

17. An assay method according to claim 1 wherein the polymerase is selected from the group consisting of a DNA polymerase, an RNA polymerase and a reverse transcriptase.

18. A modified polymerase comprising a polymerase which has been modified to include a pair of fluorophores.

19. A modified polymerase according to claim 18 wherein the polymerase is an RNA polymerase.

20. A kit comprising a modified polymerase according to claim 18 and at least one quencher group.

21. A kit according to claim 20 which comprises at least four quencher groups.

22. A kit according to claim 20 wherein the quencher group, or each quencher group, is a dark quencher group.

23. A kit according to claim 21 wherein each of the at least four quencher groups is different.

24. A kit according to claim 21, wherein each of the quencher groups includes a quencher moiety and a nucleotide which is different to the quencher moiety and nucleotide of the other quencher groups.

25. (canceled)
